(19) United States
(12) Patent Application Publication
(10) Pub. No.: US 2007/0237671 A1
(43) Pub. Date: Oct. 11, 2007

(54) Novel method of sanitizing target items using a moist sanitizing gas

(76) Inventors: James T.C. Yuan, Naperville, IL (US); Meenakshi Sundaram, Burr Ridge, IL (US); Edward F. Steiner, Downers Grove, IL (US); Steven A. Fisher, Brookfield, IL (US); Vasuhi Rasanayagam, Chicago, IL (US)

Correspondence Address: AIR LIQUIDE;Intellectual Property, 2700 POST OAK BOULEVARD, SUITE 1800, HOUSTON, TX, 77056, US

(21) Appl. No.: 11/214,344

(22) Filed: Aug. 29, 2005

(51) Int. Cl. A61L 2/18; A62B 7/08; A23B 4/023; A23L 3/3409

(52) U.S. Cl. 422/28; 422/120; 426/320; 426/264

(57) ABSTRACT

Products and methods for treating or sanitizing a target item to reduce or eliminate biological microorganisms in or on the target item. The target item is contacted with a moist sanitizing gas mixture to effectively reduce the presence of biological microorganisms without adversely affecting the quality of the target item. The moist sanitizing gas mixture of the invention is particularly useful for treating food products. The moist sanitizing gas mixture of the current invention may be formed by humidifying a dry gas and then contacting the target item with the humidified gas containing a sanitizing agent. Furthermore, the invention provides for chilling the target item, particularly food items, before exposure to the moist sanitizing gas mixture to preserve the fresh appearance, taste, and aroma of the target item.

### BACKGROUND

The reduction of microbes, spores, and other contaminants is a major concern in food, pharmaceutical, medical and other fields. Each year, economic losses for food products, due to damage from such sources, total more than $100 billion. Currently, food items are preserved using a variety of methods, including refrigeration, fumigation with toxic chemicals, irradiation, biological control, heat exposure, and controlled atmosphere storage (a fruit industry technique that involves modifying the concentration of gases naturally present in the air).

The primary problem regarding food spoilage is microbial growth. If pathogenic microorganisms are present, they can potentially lead to food-borne outbreaks and significant economic losses. Food safety concerns have been brought to the consumers' attention since the early part of the twentieth century, and those concerns have become even stronger today. Outbreaks from Salmonella and E. coli have also increased the focus on food safety. A study completed by the Centers for Disease Control and Prevention (CDC) estimated that food-borne diseases cause approximately 76 million illnesses, 325,000 hospitalizations, and 5,000 deaths annually in the United States. Those numbers reveal the dramatic need for effective means of handling food products in order to ensure food safety.

Effective sanitation depends on the combination of what is to be sanitized and the sanitation process. It is well known in the art to use biocidal agents, such as ozone, in sanitizing or treating target items, particularly food products. Biocidal agents are often used to sanitize equipment, provide antiseptic environments, and process foods. Processing foods with biocidal agents improves the safety of the food, reduces spoilage, and extends the shelf life of the food. Some biocidal agents are dissolved or absorbed in water as a mechanism to deliver the biocidal agent to a target item. Other applications feed a biocidal agent in the form of a dry gas to a treatment area to expose the target item to concentrations of sanitizing gas sufficient to significantly reduce the numbers of pathogenic organisms on the target item. Ozone is one commonly used sanitizing agent that is delivered to a treatment area of target item as a dry gas, or in an ozone-containing liquid, typically water. However, commonly used ozone-containing liquids, such as water mixtures, are limited to relatively small amounts (less than 1 mole percent ozone in water) of ozone that can be absorbed by the liquid.

A common problem in treating food items is the negative impact that some biocidal agents have on food taste and/or food color. Some agents, particularly ozone, change the color of food items; thus, the altered food item is perceived as not being fresh. Furthermore, some biocidal agents react with, or remain in the food item, altering the taste or aroma. Thus, it remains desirable to treat target items, particularly food items, using biocidal agents to reduce the number of pathogenic microorganisms, while not altering the properties, particularly the fresh appearance, taste, and aroma of the target items.

### SUMMARY

This invention addresses the need to treat, or sanitize equipment, devices, water, food or food products, or other target items using biocidal agents, while preserving the quality of the target item. The invention particularly addresses the need to be able to treat food products with biocidal agents, particularly ozone while preserving the fresh appearance, taste, and aroma of the food product. The current invention provides a process and product for sanitizing a target item, wherein a sanitizing gas mixture is humidified and then a target item is contacted with the moist sanitizing gas mixture.

In other preferred embodiments:
the target item is chilled to a temperature of about 0 to about 10° C. before being contacted with the moist sanitizing gas mixture;
the sanitizing gas mixture comprises at least about 1 wt % sanitizing agent;
the sanitizing agent is ozone, chlorine dioxide, hydrogen peroxide, chlorine, or mixtures thereof;
the moist sanitizing gas mixture comprises at least about 6 wt % said sanitizing agent;
the moist sanitizing gas mixture contains about 0.1 to about 10 wt % water;
the moist sanitizing gas mixture contains about 0.1 to about 4 wt % water;
the moist sanitizing gas mixture contains about 0.1 to about 2 wt % water;
the moist sanitizing gas mixture contains about 40 to about 60 wt % sanitizing agent;
the moist sanitizing gas mixture contains a treating gas that contains CO₂, and the sanitizing agent contains ozone;
the moist sanitizing gas mixture is humidified by a method containing the step of bubbling a treating gas through water, wherein the treating gas is a carrier gas, a sanitizing gas, or mixtures thereof;
the treating gas contains CO₂; and/or
the target item is held in a treatment area for a period of time of between about 2 seconds to about 7 minutes after exposing the target item to the moist sanitizing gas mixture.

For a further understanding of the nature and objects of the present invention, reference should be made to the following detailed description.

### DESCRIPTION

The current invention provides a process for treating a target item, particularly a food item, to reduce the presence of microbial organisms, particularly pathogenic microorganisms, in or on the target item. The current invention is particularly useful for treating food items with a biocidal agent, while preserving the fresh appearance, taste, and aroma of the food item. The current invention provides a product and method of sanitizing a target item, wherein a treating gas is humidified, a moist sanitizing gas is formed, and the target item is contacted with the moist sanitizing gas mixture. In one preferred embodiment of the invention, the moist sanitizing gas mixture comprises at least about 1 wt % sanitizing agent, and about 0.1 to about 10 wt % water. In another embodiment, the target item is preferably chilled to a temperature range of about 0 to about 10° C., and more preferably, to a temperature range of about 0 to about 5° C., before being contacted with the moist sanitizing gas.

As used herein, the phrase “target item”, refers to equipment, utensils, devices, food products, pharmaceutical products, medical devices, medical specimens, liquids, water, or other items that are in need of safe transportation, sanitation, preservation, or otherwise protecting from or treated for biological microorganisms, particularly pathogenic microorganisms.

As used herein, the phrase “food or food product”, generally refers to all types of foods, including, but not limited to, meats, poultry, seafood, produce, dry pasta, breads, cereals, and snack foods. The food may be in solid or liquid form, such as water, juice, soups, beverages, or other items. The current inventive method may be used in conjunction with any food that is able to support microbial, i.e. fungal, bacterial, or viral growth.

As used herein, the term “biocidal agent” or “sanitizing agent”, generally refers to any substance known to one of ordinary skill in the art, that when contacted with the target item reduces the number of biological microorganisms, particularly pathogenic microorganisms, on or in the target item, or reduces the growth rate of the biological microorganisms on or in the same.

The terms “sanitize” or “treat”, mean the reduction of the microbial, and/or spore content to a level such that the target item is safer to use, or safer for consumption by a mammal, particularly by humans. Target items are typically considered safer to use or consume when at least about 90.0 to about 99.9% of all microorganisms, and/or spores, including pathogenic microorganisms, in or on target items are eliminated. Thus, it is preferable to eliminate at least about 90.0 to about 99.999% of the pathogenic microorganisms, and more preferable to eliminate at least about 99.0 to about 99.999% of such microorganisms and/or spores.

The current inventive method uses a moist sanitizing gas mixture to treat target items, or to be fed to treatment areas to sanitize a target item. The moist sanitizing gas mixture provides a greater reduction of pathogenic microorganisms than a dry sanitizing gas mixture. In particular, treating target items with a moist sanitizing gas mixture containing ozone as a sanitizing agent provides significantly greater reduction of pathogenic microorganisms than treating target items with a dry ozone gas mixture. The sanitizing gas mixture can be introduced to the treatment area substantially concurrent with other treating materials, such as air, CO₂, N₂O, N₂, Ar, He, and mixtures thereof.

The moist sanitizing gas mixture of the current invention is supplied to the treatment area as a gas that contains water. The moist sanitizing gas mixture can contain any amount of water that the particular gas mixture can support in the gas phase. Preferred moist sanitizing gas mixtures contain between about 0.1 and about 10 wt % water, between about 0.1 and about 4 wt % water, or between about 0.1 and about 2 wt % water. Thus, the moist sanitizing gas mixture provides significant benefit over traditional processes using mixtures of water containing ozone because it is not necessary to feed, collect, and recycle large volumes of water in the process.

The water used in the current process can be any water suitable for contact with the target item. The water is preferably filtered, deionized, distilled, or otherwise treated to remove unwanted contaminants or components. Because the water contacts a sanitizing agent before contacting the target item, the process also sanitizes the water before it contacts the target item.

The moist sanitizing gas mixture of the current invention contains a sanitizing agent. The sanitizing agent can be any biocidal agent known to one skilled in the art that is effective in reducing the number of biological microorganisms, particularly pathogenic microorganisms, on or in the target item, or reduces the growth rate of the biological microorganisms on or in the same, and is compatible with water. Preferred sanitizing agents include, but are not limited to, ozone, chlorine dioxide, hydrogen peroxide, chlorine, and mixtures thereof. The moist sanitizing gas mixture preferably contains at least about 1 wt % sanitizing agent, more preferably, greater than about 2 wt % sanitizing agent, and even more preferably, greater than about 6 wt % sanitizing agent. One embodiment of the moist sanitizing gas contains between about 40 and 60 wt % sanitizing agent.

The current process contacts a target item with the moist sanitizing gas mixture to sanitize the target item. In one preferred embodiment, the moist sanitizing gas mixture is fed into a treatment area containing the target item in order to contact the target item. The treatment area may be any one of a wide variety of vessels or equipment used to process a target item. Examples for treatment areas that process food products include, but are not limited to, a tunnel, tumbler, blender, plate, chamber, vessels, and combinations of these devices. Other embodiments may involve feeding the moist sanitizing gas into the target item, such as into an animal carcass, or into equipment that is to be sanitized, such as lines, vessels, or other processing devices.

The moist sanitizing gas may be formed by any method effective in creating the desired moist sanitizing gas mixture. As used herein, humidifying a treating gas means combining water and a treating gas to form a gaseous mixture that contains water. The treating gas is preferably a gaseous form of the sanitizing agent, a gaseous mixture containing the sanitizing agent, or a carrier gas that does not contain the sanitizing agent. A carrier gas is preferably any gas that is compatible with the target item, and can be used to transport the water and sanitizing agent to the target item. In one embodiment, the carrier gas is an inert gas, such as nitrogen, or argon. In another embodiment, the carrier gas is a gas that is reactive with the target item, such as a ozone containing gas mixture, or CO₂. In embodiments where the carrier gas does not contain the sanitizing agent, the water may be combined with the carrier gas before or after the sanitizing agent is combined with the carrier gas. One preferred means of humidifying a treating gas is bubbling the treating gas up through a reservoir of water. When the treating gas bubbles up through the water, it picks up water in the gas phase of the treating gas. The treating gas then exits as a humidified gas. In one embodiment, the treating gas is saturated with water.

In one embodiment of forming a moist sanitizing gas, a CO₂ carrier gas is bubbled up through a reservoir of water to humidify that CO₂ carrier gas, which is then combined with an ozone-containing stream to form the moist sanitizing gas mixture. In a second embodiment, an ozone-containing gas stream is bubbled through water to humidify the ozone-containing gas stream, thus forming the moist sanitizing gas mixture. In a third embodiment, a sanitizing agent is combined with a carrier gas to form a dry sanitizing gas mixture, which is then humidified by combing the dry sanitizing gas mixture with water. In this embodiment, the dry sanitizing gas mixture is humidified by bubbling the dry sanitizing gas mixture through water, or by adding water to the dry sanitizing gas mixture.

In another preferred embodiment of the current invention, the target item is chilled before being contacted with the moist sanitizing gas. Where the target item is a food item, chilling the target item before being contacted with the sanitizing agent improves the appearance, taste, and aroma of the food item, while maintaining an effective reduction in pathogenic microorganisms. Many food items, particularly fish and chicken, change their appearance by changing color after exposure to some sanitizing agents, particularly ozone. By chilling the food item to a range of about 0 to about 10° C., and more preferably to a range of about 0 to about 5° C., before the sanitizing agent contacts the food item, the food item retains its fresh color, even after exposure to the sanitizing agent. Furthermore, taste testing has shown that the food items exposed to the sanitizing agent, particularly ozone, while the food item is above about 10° C., take on a taste attributable to the sanitizing agent that is objectionable to consumers. By chilling the target item before exposure to the sanitizing agent, the taste and aroma of the target item does not change, or the change is minimal.

To optimize the effectiveness of the sanitation process, it is desirable to maintain contact between the target item and the moist sanitizing gas for a period of time. In one embodiment, the target item is held in a treatment area for a period of time ranging from about 2 seconds to about 7 minutes. The optimum hold time may be easily determined by experiments involving the particular sanitizing agent, target item, and holding conditions.

One embodiment of the current invention uses ozone as the sanitizing agent. Methods of producing ozone are well known in the art. Ozone can be generated using oxygen or air. Two primary methods of creating ozone from air are by an ultraviolet light generator light system or by an electrical discharge system. An ultraviolet light ozone generator typically consists of multiple ultraviolet light tubes within a aluminum housing. In a multiple tube apparatus, air enters the generator cavity and is subjected to the ultraviolet light, and the ultraviolet light causes a disassociation of the oxygen molecules, which exists as O₂, to two oxygen atoms. Some of these oxygen atoms attach themselves to oxygen molecules to form ozone (O₃). The resulting ozone and sterile air mixture comprises approximately 0.2 percent of ozone by weight/weight of air. In one preferred mode, the ozone gas is generated from oxygen or oxygen-enriched air by a corona discharge device that produces concentrations ranging between about 1% to about 15% by weight of ozone. Based on technologies available today, it is possible to generate ozone concentrations up to about 13.5% with the remainder being oxygen and a small fraction of other gases. It is possible to use higher ozone concentrations for this application if the generator technology becomes available. Higher concentrations of ozone are preferred. It is also preferred to use oxygen compared to air due to the possibility of producing higher concentrations of ozone.

Small amounts of adjuvants may be added into the moist sanitizing gas mixture to improve the stability of the sanitizing agent in the mixture, or provide other desirable effects on the target item.

### EXAMPLES

The current invention will now be described in terms of non-limiting examples, wherein ozone was used as the sanitizing agent for all treated samples.

### Example 1

Example 1 demonstrates the beneficial effect of moisture in reducing pathogenic microorganisms on a target item. L. monocytogenes was inoculated on stainless steel coupons and placed in a closed, dry chamber. Either a moist or a dry CO₂/ozone mixture was introduced to the chamber and held for about two hours. The ozone was generated and mixed with a CO₂ stream to dilute to 10 ppmv ozone content. This O₃/CO₂ stream was bubbled through a column of deionized water at 4° C. in a “gas washing bottle”. This humidified gas stream was then sent through an additional empty glass vessel to remove entrained water droplets. The humidified gas was then directed through flow meters and then to the coupon chambers. By calculation, it was estimated the humidified gas contained about 0.5 wt % water. The molecular weight of the gas mixture was about 43 g/gmole in the coupon chamber.

Results of Table 1 illustrate that dry ozone did not provide an effective reduction in the level of pathogenic microorganisms. For the same ozone concentration and temperature, about one log reduction was observed in the moist atmosphere.

TABLE 1

| Treatment | Moisture | Microbial reduction |
|---|---|---|
| Temperature | condition | (In log scale) |
| ˜4° C. | Dry | <0.2 |
| ˜4° C. | Moist | ˜2.5 |

Treatment of stainless steel coupons inoculated with L. monocytogenes.

### Example 2

Example 2 demonstrates the favorable effect on appearance and taste of a food item achieved by chilling the food item before exposing the food item to the moist sanitizing agent.

Boneless, skinless chicken breast samples were obtained. A sensory control group was placed in 24-oz sterile Whirl-pak plastic sampling bags (Nasco, Fort Atkinson, Wis.) and kept at 2° C. until sensory analysis was performed. The remaining samples were inoculated with 0.2 ml of Salmonella enteritidis and allowed to air dry for about 30 minutes. The inoculation was performed by the spread method on one side of the chicken. Inoculated chicken was divided into a microbial control group and a treatment group. The microbial control group pieces received no further treatment, and were transferred into 24-oz sterile Whirl-pak plastic sampling bags (Nasco, Fort Atkinson, Wis.) and kept at 2° C. until microbiological analysis was performed.

The treatment group of inoculated chicken was divided into two groups, a group that was chilled before treatment with a moist sanitizing gas mixture, and a group that was treated with a moist sanitizing gas mixture without chilling. The moist sanitizing gas mixture contained ozone as the sanitizing agent. Experiments were carried out in batch reactors.

The initial temperature for both groups was about 20° C. For the chilled treatment group, the reactor was cooled first (without chicken) using CO₂ snow to about −20° C. The chilled treatment group was placed in the cold reactor and cooled to a target temperature of about 0 to about 5° C. using pre-measured CO₂ snow. Following the cooling step, a moist sanitizing gas mixture (CO₂/ozone/water) was established in the reactor. The CO₂ was made moist by bubbling it through a stainless steel “gas washing bottle”, where the deionized water was held at about 30° C. The moist CO₂ was added to the treatment chamber to achieve a pressure of about 50 psig. Pressurized O₃ was added to the treatment chamber to achieve 200 milligrams of ozone per kilogram of chicken. The moist sanitizing gas mixture was maintained in the reactor for five minutes and released. By calculation, the equivalent amount of water in the sanitizing gas mixture was determined to be about 2.8 wt %. The molecular weight of the moist sanitizing gas mixture was about 41 g/gmole in the reactor. The same experimental steps were followed for the un-chilled treatment group, except the cooling steps were excluded. Thus, the un-chilled treatment group was treated at a temperature of about 19 to about 20° C.

Control (inoculated) and treated samples (chilled and un-chilled) were stomached using Seward's Laboratory Blender, Stomacher 400, speed set at “High” for 2 minutes with 90 ml sterile peptone water. The samples were serially diluted and plated on xylose-lysine-desoxycholate agar (XLD, Difco). The plates were held at 35° C. for one day. The efficacy of the treatment was determined as the difference of the microbial counts between control and treated samples.

The same experimental procedures described above were used for evaluating the effect of moist ozone treatment on the sensory qualities of the samples using un-inoculated chicken. The treated (chilled and un-chilled) samples, and control samples were compared visually against the control samples for changes in color before cooking. The samples were then marinated with pepper and salt, grilled, and evaluated for sensory quality. That is, the treated samples (chilled and un-chilled) were compared against the control samples for changes in taste. Results illustrate that even though treatment at the higher temperature moderately increased the microbiological inactivation, it significantly and negatively affected the quality of the chicken as measured by visual appearance and taste. Table 2 shows the microbial reduction, visual inspection, and sensory evaluation (taste and aroma) of the treated chicken samples as compared to the control samples. Clearly, the samples treated with the moist sanitizing gas at cold temperatures resulted in a more desirable food product.

TABLE 2

| | Ozone | | | |
|---|---|---|---|---|
| | con- | | | |
| | centration | Microbial | | |
| Treatment | (mg/kg of | reduction | Quality | |
| Temperature | chicken) | (in log scale) | Color | Sensory |
| 19-20° C. | 200 | 1.34 | Discoloration | Cooked |
| | | | | chicken |
| | | | | smelled and |
| | | | | tasted ozone |
| 1-2° C. | 200 | 1.08 | No color | No or minimal |
| | | | change | taste |
| | | | | difference |

Evaluation of chicken samples after treatment with moist sanitizing gas mixture.

### Example 3

Example 3 demonstrates the effect of moist ozone treatment on food items compared to the effect of CO₂ alone. The same procedure was followed as outlined above for Example 2, except during the gas injection, one group was treated with CO₂ only, and one group was treated with CO₂ and moist ozone.

As shown in Table 3, CO₂ alone did not significantly reduce the microbial load on the chicken pieces. Adding moist ozone reduced the microbial load by 90%, while preserving the color and sensory qualities of the chicken.

TABLE 3

| | | Microbial | | |
|---|---|---|---|---|
| Treatment | | reduction | Quality | |
| Temperature | Gas Environment | (in log scale) | Color | Sensory |
| 1-2° C. | CO₂ only | 0.1-0.02 | No color | No or |
| | | | change | minimal |
| | | | | taste |
| | | | | difference |
| 1-2° C. | CO₂/ozone/water | 1.08 | No color | No or |
| | mixture | | change | minimal |
| | (200 mg of | | | taste |
| | ozone/kg of | | | difference |
| | chicken) | | | |

Effect of CO₂ and moist ozone at chilled conditions on the microbiological and sensory quality of chicken.

### Example 4

Example 4 demonstrates a commercial scale trial of the moist sanitizing gas mixture process. Sample preparation was followed as explained in Example 2. A commercial freezer (MBI Cryogenics, Ballwin, Mo.) was used as the treatment area for contacting target items (chicken) with a moist sanitizing gas mixture, wherein ozone was the sanitizing agent. The freezer was cooled to around −40° C. with liquid and gaseous CO₂ before starting the experiment. After cooling the freezer, the shelving unit was pulled out and chicken pieces were loaded on the trays. Temperatures of the chicken breasts were measured at three locations on each piece during the treatment along with the temperature inside the freezer. Two fans in the freezer circulated the moist sanitizing gas mixture within the freezer. When the surface temperature of all three chicken pieces was about 2-3° C., cooling was stopped, and a moist CO₂/ozone gas mixture was injected. The ozone concentration inside the chamber was monitored. After the ozone concentration inside the freezer reached about 1 wt %, it was held at that concentration for 90 seconds. The ozone was then evacuated and the chicken pieces were transferred into sterile Whirl-pak plastic sampling bags for microbiological analysis. The same experiment was repeated on chicken pieces without inoculation for the sensory analysis.

In this example, ozone was generated to about 8.5 wt % ozone in oxygen. CO₂ was humidified by bubbling it through a stainless steel “gas washing bottle”, containing de-ionized water held at 35° C. The ozone and CO₂ gas streams were combined to make a 5.0 wt % ozone in the mixture. The mixture was fed to the food freezer to achieve the 1 wt % ozone in the freezer. In this example, the amount of water in the CO₂ was estimated to be about 3.7 wt %, which was diluted by the ozone in the feed gas to become about 2.4 wt % in the sanitizing gas mixture. This was further diluted by the volume of gas in the freezer to become about 0.5 wt % water in the atmosphere contained in the freezer. The molecular weight of the gas mixture in the freezer was about 44 g/gmole.

Table 4 shows the results reflecting the same trend from two replications of the experiment for moist sanitizing gas treatment, as indicated in the examples above when implemented on the industrial scale system. Pathogenic microbial load was reduced by 90% without significantly changing the quality of the chicken.

TABLE 4

| | Ozone | | | |
|---|---|---|---|---|
| | con- | | | |
| | centration | Microbial | | |
| Treatment | (% by | reduction | Quality | |
| Temperature | weight) | (In log scale) | Color | Sensory |
| 0-5° C. | 1˜1.07 | 0.99 | No color | Minimal taste |
| | | | change | difference |
| 0-5° C. | 1˜1.1 | 1.05 | No color | Minimal taste |
| | | | change | difference. |

Effect of moist ozone on the microbiological and sensory quality of chicken at chilled conditions in an industrial scale freezer.

Although the present invention has been described in considerable detail with reference to certain preferred versions and examples thereof, other versions are possible. For instance, the sanitizing agent of the current invention can be any sanitizing agent that benefits from the addition of moisture and chilling before treatment. Furthermore, the current invention may be used in a variety of processes for sanitizing food, or non-food items. Therefore, the spirit and scope of the appended claims should not be limited to the description of the preferred versions contained herein.

What is claimed is:

1. A method of sanitizing a target item comprising the steps of:
a) forming a moist sanitizing gas mixture, wherein said moist sanitizing gas mixture comprises a sanitizing agent, and wherein said moist sanitizing gas mixture is formed by a method comprising a step of humidifying a treating gas; and
b) contacting a target item with said moist sanitizing gas mixture.

2. The method of claim 1, wherein said sanitizing agent is selected from the group consisting of:
a) ozone;
b) chlorine dioxide;
c) hydrogen peroxide;
d) chlorine; and
e) mixtures thereof.

3. The method of claim 2, wherein said treating gas comprises:
a) ozone;
b) air;
c) carbon dioxide;
d) nitrous oxide;
e) nitrogen;
f) argon;
g) helium; and
h) mixtures thereof.

4. The method of claim 3, further comprising the step of chilling said target item to a temperature of about 0 to about 10° C. before said contacting step.

5. The method of claim 4, wherein said humidifying step further comprises a step of bubbling said treating gas through a reservoir of water.

6. The method of claim 5, wherein said moist sanitizing gas mixture comprises at least about 6 wt % said sanitizing agent.

7. The method of claim 1, wherein said moist sanitizing gas mixture comprises:
a) at least about 1 wt % sanitizing agent; and
b) water, wherein said water is in a range of about 0.1 to about 10 wt % of said moist sanitizing gas mixture.

8. The method of claim 7, further comprising the step of chilling said target item to a temperature of about 0 to about 10° C. before said contacting step.

9. The method of claim 8, wherein said water is in a range of about 0.1 to about 4 wt % of said moist sanitizing gas mixture

10. The method of claim 9, wherein said sanitizing agent is selected from the group consisting of:
a) ozone;
b) chlorine dioxide;
c) hydrogen peroxide;
d) chlorine; and
e) mixtures thereof.

11. The method of claim 10, wherein said moist sanitizing gas mixture comprises at least about 6 wt % said sanitizing agent.

12. The method of claim 11, wherein said moist sanitizing gas mixture comprises about 40 to about 60 wt % said sanitizing agent.

13. The method of claim 12, wherein said treating gas comprises carbon dioxide, and wherein said sanitizing agent comprises ozone.

14. A method of sanitizing a target item comprising the step of contacting a target item with a moist sanitizing gas mixture, wherein said moist sanitizing gas mixture comprises:
a) at least about 1 wt % sanitizing agent; and
b) water, wherein said water is in a range of about 0.1 to about 10 wt % of said moist sanitizing gas mixture.

15. The method of claim 14, wherein said moist sanitizing gas mixture comprises at least about 6 wt % sanitizing agent.

16. The method of claim 14, wherein said water is in a range of about 0.1 to about 4 wt % of said moist sanitizing gas mixture.

17. The method of claim 14, further comprising the step of chilling said target item to a temperature range of about 0 to about 10° C.

18. The method of claim 17, wherein said sanitizing agent is selected from the group consisting of:
a) ozone;
b) chlorine dioxide;
c) hydrogen peroxide;
d) chlorine; and
e) mixtures thereof.

19. The method of claim 18, wherein said sanitizing agent comprises ozone.

20. The method of claim 14, further comprising the step of forming said moist sanitizing gas mixture, wherein said forming step comprises a step of bubbling a treating gas through water, wherein said treating gas is selected from a group consisting of a carrier gas, a sanitizing gas, and mixtures thereof.

21. The method of claim 20, wherein said treating gas comprises carbon dioxide.

22. The method of claim 21, wherein said sanitizing agent comprises ozone.

23. The method of claim 22, further comprising a step of holding said target item in a treatment area for a period of time of between about 2 seconds to about 7 minutes after exposing said target item to said moist sanitizing gas mixture.

24. A moist sanitizing gas mixture comprising:
a) at least about 1 wt % sanitizing agent; and
b) water, wherein said water is in a range of about 0.1 to about 10 wt % of said moist sanitizing gas mixture.

25. The moist sanitizing gas mixture of claim 24, wherein said sanitizing agent is selected from the group consisting of:
a) ozone;
b) chlorine dioxide;
c) hydrogen peroxide;
d) chlorine; and
e) mixtures thereof.

26. The moist sanitizing gas mixture of claim 25, wherein said moist sanitizing gas mixture comprises at least about 6 wt % said sanitizing agent.

27. The moist sanitizing gas mixture of claim 25, wherein said water is in a range of about 0.1 to about 4 wt % of said moist sanitizing gas mixture.

28. The moist sanitizing gas mixture of claim 25, wherein said moist sanitizing gas mixture comprises about 40 to about 60 wt % said sanitizing agent.

29. The moist sanitizing gas mixture of claim 25, wherein said moist sanitizing gas mixture further comprises carbon dioxide.
